(19) United States
(12) Patent Application Publication
(10) Pub. No.: US 2009/0191247 A1
(43) Pub. Date: Jul. 30, 2009

(54) Anti-Viral Uses Of Carbon And Metal Nanomaterial Compositions

(75) Inventors: Miguel Jose-Yacaman, San Antonio, TX (US); Kurt A Schroder, Coupland, TX (US); Karl M. Martin, Austin, TX (US); Darrin L. Willauer, Pelugerville, TX (US)

Correspondence Address: Dillon & Yudell, LLP, 8911 N. Capital Of Texas Highway, Suite 2110, Austin, TX, 78759, US

(73) Assignee: Nanotechnologies, INC, Austin, TX (US)

(21) Appl. No.: 11/792,433

(22) PCT Filed: Dec. 5, 2005
(86) PCT No.: PCT/US05/43686

§ 371 Date: Feb. 10, 2008

Related U.S. Application Data
(60) Provisional application No. 60/633,671, filed on Dec. 6, 2004.

(51) Int. Cl. A01N 25/08; A01N 59/16; A01N 59/00; A01N 25/00; A61K 33/38; A01N 25/26

(52) U.S. Cl. 424/402; 424/618; 424/125; 424/405; 424/421; 977/810

(57) ABSTRACT

This invention generally relates to use of novel nanomaterials comprised of metals in anti-viral applications. Such nanomaterials, for example, can be produced using a high power, pulsed plasma process, which plasma process, optionally, can be performed on the metal with a precursor (i.e., a gaseous precursor, such as acetylene or methane) when forming the unagglomerated nanomaterials. In embodiments of the invention, the metal is nanosilver. Optionally, the nanomaterials may also comprise carbon, including in the form of carbyne.

### RELATED PATENT APPLICATIONS

This patent application claims the benefit of the earlier filing date of U.S. Patent Application No. 60/633,671 (filed Dec. 6, 2004), which application is entitled “Anti-Viral Uses of Carbon and Metal Nanomaterial Compositions,” having Miguel Jose Yacaman, Kurt A. Schroder, Schroder, Karl Mathew Martin, and Darrin L. Willauer, as inventors. This application is assigned to the Assignees of the present invention.

This application is also related to the following patent applications:

PCT Patent Application No. PCT/US2005/027711, filed Aug. 4, 2005, entitled “Carbon And Metal Nanomaterial Composition And Synthesis” having Kurt Schroder and Karl Matthew Martin as inventors (“the PCT 05/027711 Application”), and claiming benefits of the earlier filing dates of U.S. Patent Application Nos. 60/598,784 (filed Aug. 4, 2005) and 60/620,181 (filed on Oct. 19, 2004), which two provisional patent applications have the same title and named inventors as the PCT 05/027711 Application.

U.S. patent application Ser. No. 10/669,858, filed on Sep. 24, 2003, entitled “Nanopowder Synthesis Using Pulsed Arc Discharge and Applied Magnetic Field,” having Kurt Schroder and Doug Jackson as inventors (“the '858 patent Application”).

U.S. Pat. No. 6,777,639, issued on Aug. 17, 2004, entitled “Radial Pulsed Arc Discharge Gun For Synthesizing Nanopowders,” having Kurt Schroder and Doug Jackson as inventors.

Each of the applications and patent identified above are incorporated herein by reference.

### FIELD OF THE INVENTION

This invention generally relates to use of novel nanomaterials comprised of metals in anti-viral applications. Such nanomaterials, for example, can be produced using a high power, pulsed plasma process, which plasma process, optionally, can be performed on the metal with a precursor (i.e., a gaseous precursor, such as acetylene or methane) when forming the unagglomerated nanomaterials. In embodiments of the invention, the metal is nanosilver. Optionally, the nanomaterials may also comprise carbon, including in the form of carbyne.

### BACKGROUND

In the field of antimicrobial agents, many compounds are used. Traditionally, antibiotics are used to kill bacteria by disrupting the bacteria's respiratory functions, disrupting its metabolic functions, such as protein synthesis or cell wall formation, or blocking the formation of the bacteria's DNA or RNA. In all these cases the antibiotic works by disrupting or blocking some of the living cell's function. One material that has long been used as an antibacterial agent is silver. It was used by the early Roman's to purify water and is currently used in the medical field as a broad spectrum antibiotic. The mechanism by which the silver works has been studied in great depth. The general consensus is that silver works by releasing silver ions to disrupt the cell wall production and inhibits the DNA production The publication by Q. L. Feng, J. Wu, G. Q. Chen, F. Z. Cui, T. N. Kim, J. O. Kim, “A mechanistic study of the antibacterial effect of silver ions on Escherichia coli and Staphylococcus aureus,” Journal of Biomedical Materials Research, Volume 52, Issue 4, Pages 662-668, Oct. 3, 2000 discusses many of the details of these mechanism.

Westaim Technologies, Inc. also utilized silver as an antibacterial agent by using silver that contains nanoscale structures, such as discussed in U.S. Pat. Nos. 5,837,275, 5,454,886, and 5,958,440. In that case, the high surface area to volume ratio of the silver relative to bulk micron silver appears to give better ion release than the larger sized silver. Additionally, Westaim Technologies specifically discussed that it created defects in the nanostructures to improve the ionic release of the silver. This silver antibacterial agent is used in many different applications ranging from wound dressings to coatings on medical devices.

Recently, Sondi and Salopek-Sondi published a paper, “Silver nanoparticles as antimicrobial agent: a case study on E. coli as a model for Gram-negative bacteria,” Journal of Colloid and Interface Science 275 (2004), pages 177-182, that indicates that nanoparticles of silver may also have a different mechanism by which it kills bacteria. In their research, they found the smaller nanoparticles have direct interaction with the bacteria, however the exact mechanism of the kill was undetermined. In their testing, they showed that the silver particles penetrated the cell wall which eventually killed the bacteria.

In all these antimicrobial agents, the mechanism by which the kill occurs was the disruption of the cell's bioactivity. For this reason, antimicrobial agents do not work on viruses. Viruses are not living organisms but are pieces of nucleic acid (DNA or RNA) wrapped in a thin coat of protein. They attach to another micro-organism, such as a bacteria or a cell, which causes a series of events that transforms the host organism such that the host organism begins reproducing more viruses within itself until the host organism ruptures and releases the new viruses.

The immune system responds to viruses by producing antibodies that bind to the virus so that the virus can not bind to the host cell. Hence for each virus there must be a specific antibody. This makes creating an antiviral agent very difficult because of the numerous permutations of DNA structures in viruses. Additionally, the immune system will often respond to a virus by increasing the core body temperature to kill the virus. Hence there is a need for an antiviral agent that has broad spectrum kill that can kill the virus without heat.

### SUMMARY OF THE INVENTION

This invention generally relates to uses of nanomaterials comprised of metals used as an anti-viral agent. For example, the high power, pulsed plasma processes described in the PCT 05/027711 Application and the '858 patent Application produce materials comprising nanometals. (In the current invention, nano refers to a material having dimensions less than about 1 micron. Generally, the dimensions are less than about 500 nm, and even more so less than about 100 nm). In embodiments of the invention, the metal can be nanosilver. Furthermore, the nanomaterials may further comprise carbon, including in the form of a carbyne. Such carbon may be included within the nanomaterials by utilizing a precursor (i.e., a gaseous precursor, such as acetylene or methane) during a high power, pulsed plasma process when formiing the nanomaterials. The nanomaterials utilized in embodiments of the invention have a combination of attributes and properties that allow them to be used for anti-viral applications.

One embodiment of the current invention uses a nanosized silver/carbon composite as an anti-viral agent. In some embodiments, the composition is in the form of a nanopowder with an average size of less than about 25 nm and in further embodiments, the average is less than about 8 nm.

Because all the current literature teaches that the silver antibacterial properties are linked to the bacteria's bioactivity, the literature suggests that silver would not be an effective anti-viral agent. Nonetheless, this nanosized silver/carbon composite has shown to be quite effective as an anti-viral agent.

### BRIEF DESCRIPTION OF THE DRAWINGS

FIG. 1 is a TEM image of a 77 nm silver composition.

FIG. 2 is a TEM image of a 45 nm silver/carbon composition.

FIG. 3 is a TEM image of a 30 nm silver/carbon composition.

FIG. 4 is a TEM image of a 28 nm silver/carbon composition.

FIGS. 5A-B are TEM images of a 25 nm silver/carbon composition.

FIG. 6 is a TEM image of a 22 silver/carbon composition.

FIGS. 7A-C are TEM images of a 10 nm silver/carbon composition.

FIG. 8 is a TEM image of the 10 nm silver/carbon composition also shown in FIGS. 7A-7C.

FIG. 9 is a TEM image of a 9 nm silver/carbon composition.

FIGS. 10A-F are TEM images of carbon/silver compositions.

FIGS. 11A-C are TEM images of a silver/carbon composition, which shows the presence of carbyne.

FIGS. 12A-D are TEM images of a copper/carbon composition, which shows the presence graphitic and fullerene carbon.

FIGS. 13A-B are TEM images of an iron/carbon composition, which shows the presence graphitic and fullerene carbon.

FIGS. 14A-B are TEM images of an iron/silver/carbon composition/alloy.

FIG. 15 is a bar graph reflecting syncitia percentage in MT-2 by HIV-1 exposed to a 25 nm silver/carbon composition at varying concentrations.

FIG. 16 is a TEM image of a silver/carbon composition irradiated by an electron beam.

### DETAILED DESCRIPTION

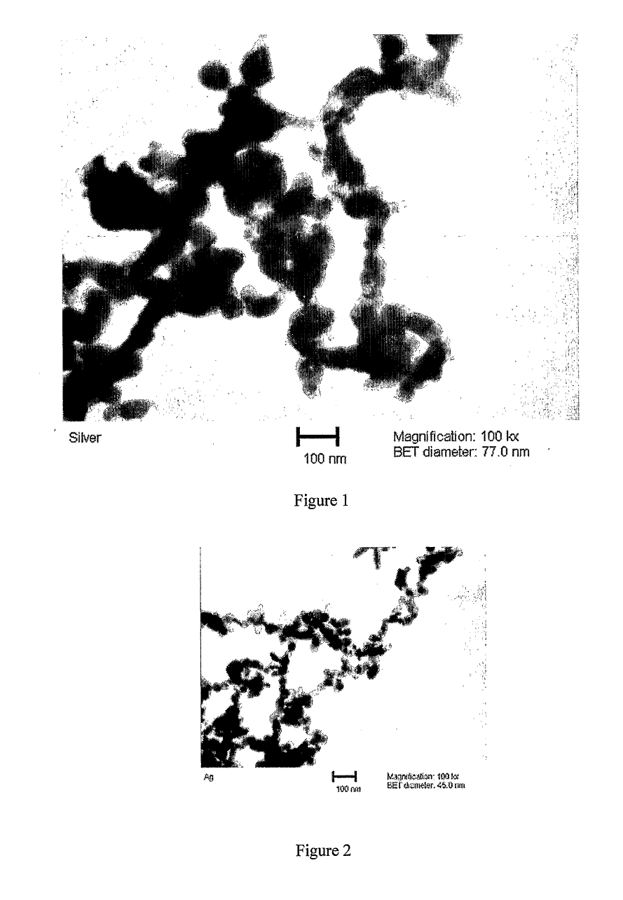

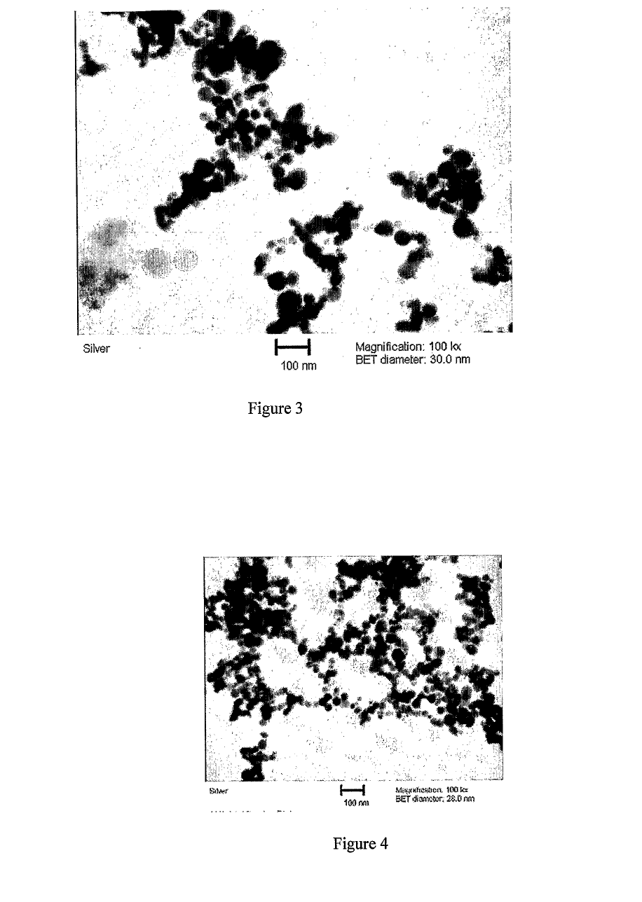

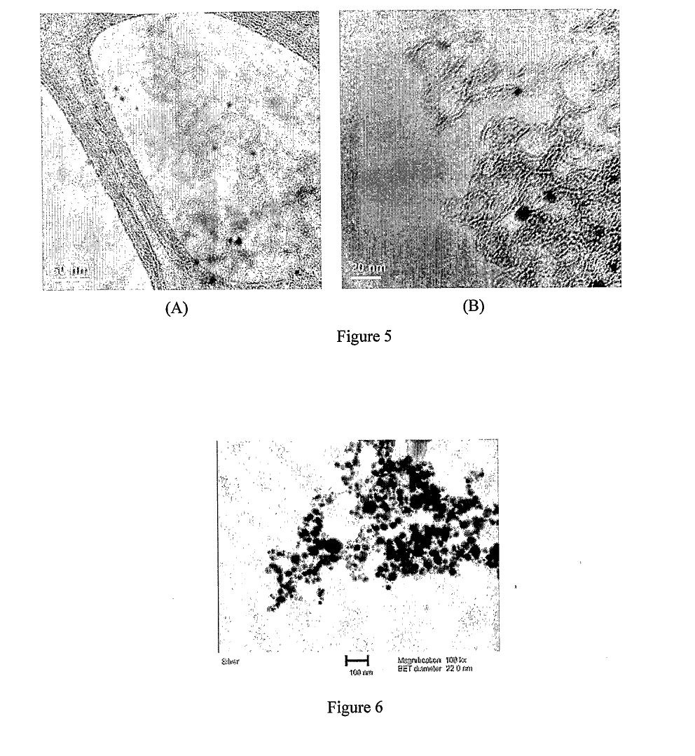

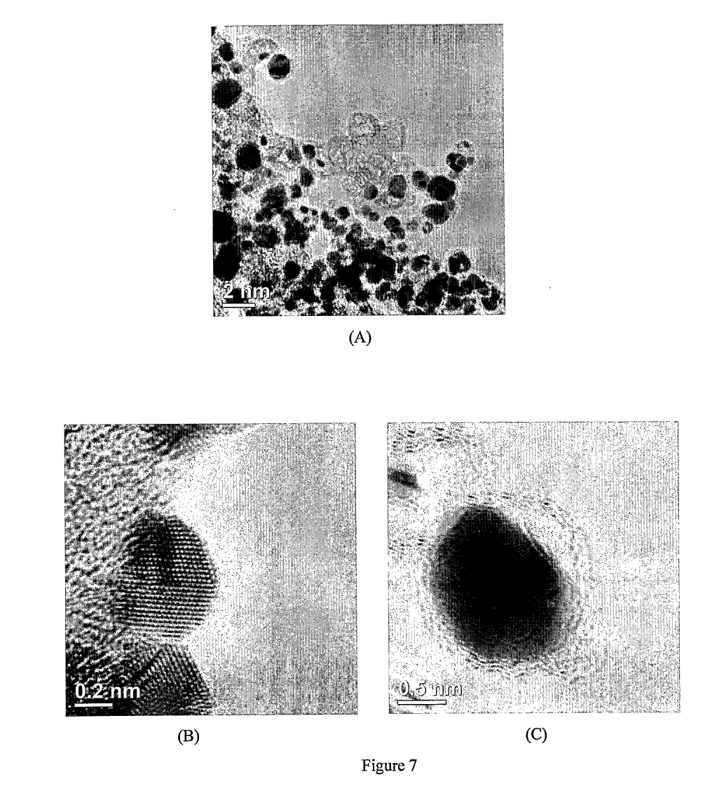

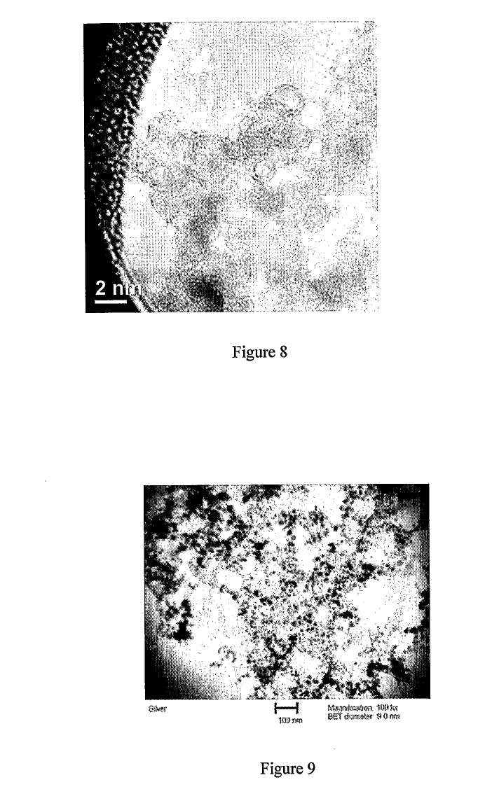

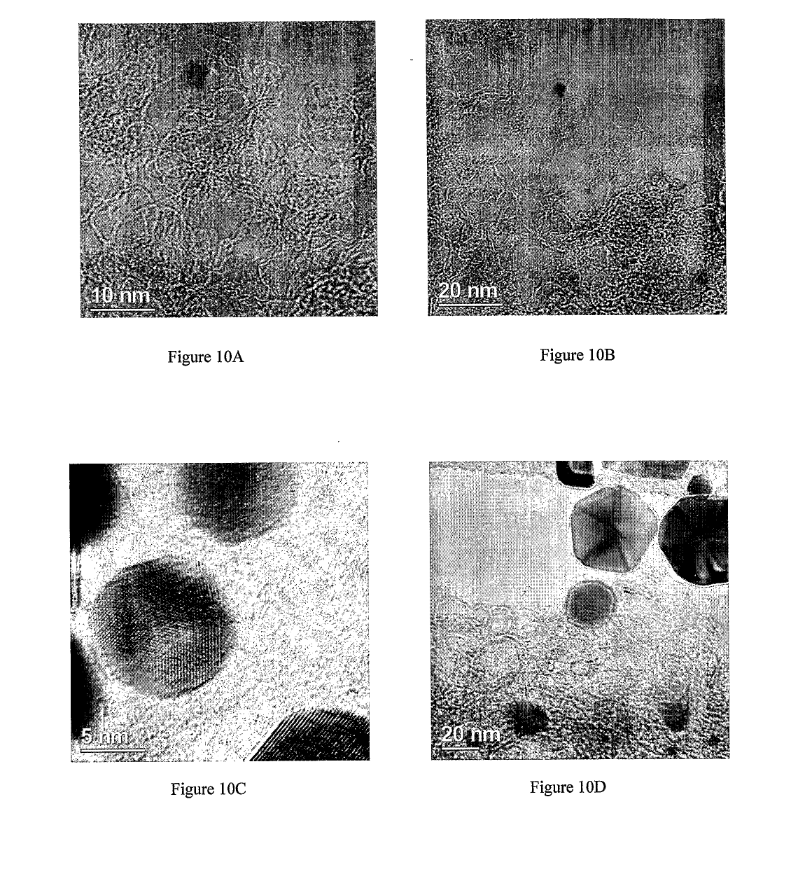

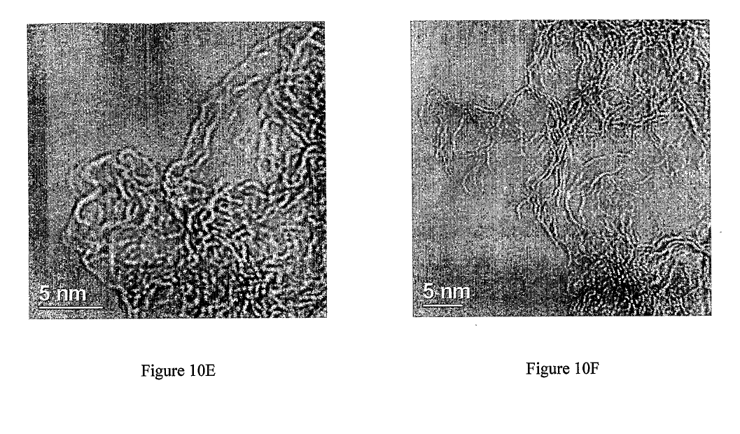

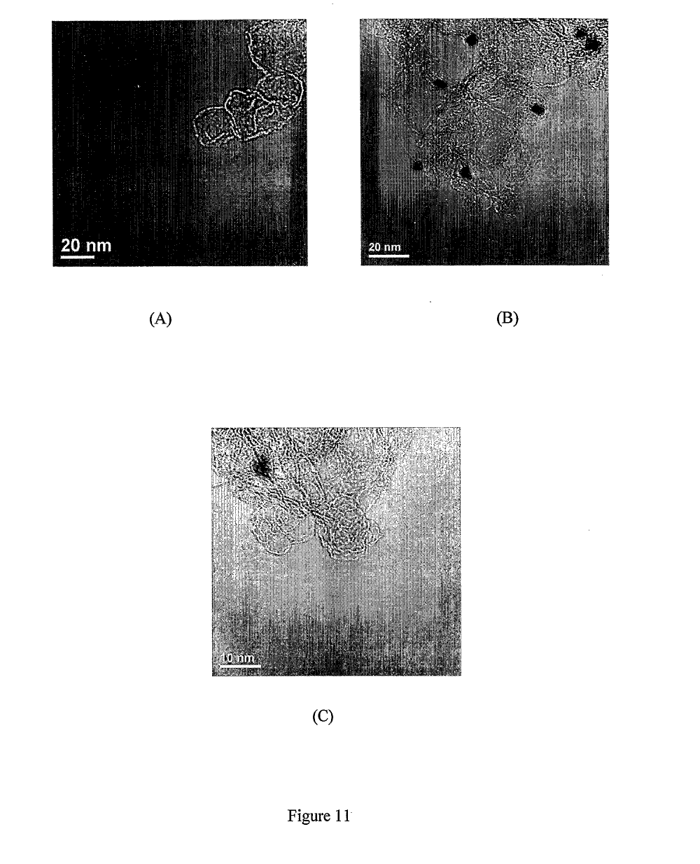

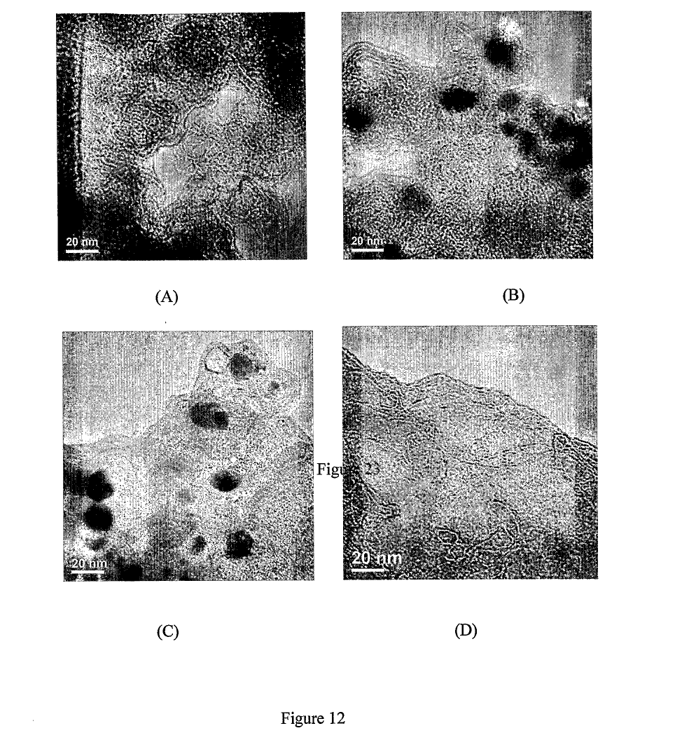

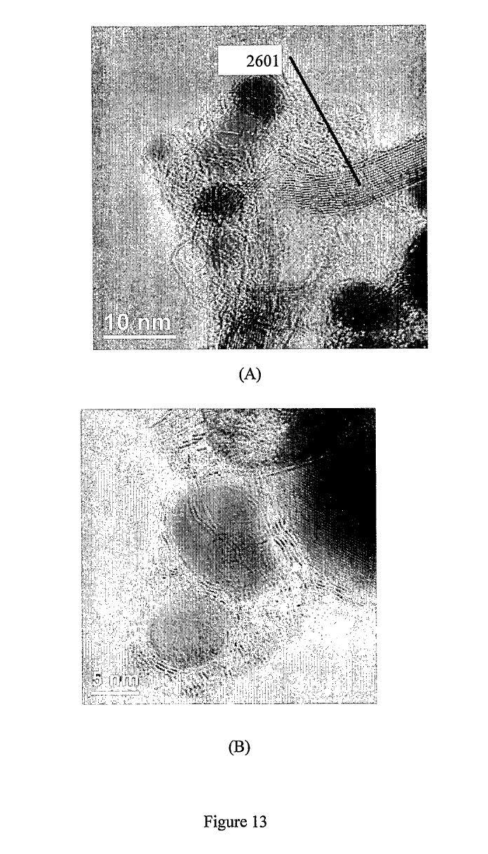

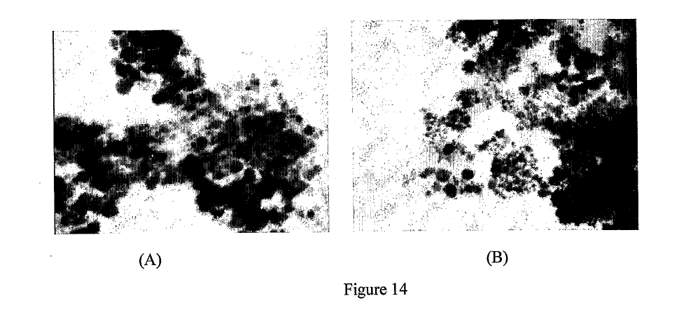

Nanometal materials can be utilized to exploit their unique properties. Such nanomaterials include those made by the processes described in the PCT 05/027711 Application and the '858 patent Application. Examples of nanometals that can be utilized in the present invention include:
(a) 77 nm silver composition produced with no hydrocarbon gas, utilizing the process described in Example 1 of the PCT 05/027711 Application. FIG. 1 shows this produced material.
(b) 45 nm silver/carbon composition produced by adding a 44-ppm concentration of acetylene, utilizing the process described in Example 1 of the PCT 05/027711 Application. FIG. 2 shows this produced material.
(c) 30 nm silver/carbon composition produced by using a 8800-ppm concentration of methane, utilizing the process described in Example 1 of the PCT 05/027711 Application. FIG. 3 shows this produced material.
(d) 28 nm silver/carbon composition produced by adding a 440-ppm concentration of acetylene, utilizing the process described in Example 1 of the PCT 05/027711 Application. FIG. 4 shows this produced material.
(e) 25 nm silver/carbon composition produced by adding a 400-ppm concentration of acetylene, utilizing the process described in Example 1 of the PCT 05/027711 Application. This composition has a specific surface area of 22 m2/g (BET) and is 97 wt. % of silver and 3 wt. % carbon. FIGS. 5A-B show this produced material.
(f) 22 nm silver/carbon composition produced by adding a 4,400-ppm concentration of acetylene, utilizing the process described in Example I of the PCT 05/027711 Application. FIG. 6 shows this produced material.
(g) 10 nm silver/carbon composition produced by adding a 44,000-ppm concentration of acetylene, utilizing the process described in Example 1 of the PCT 05/027711 Application. This process yielded a material with a specific surface area of 60 m2/g that was 70 wt. % silver and 30 wt. % carbon. The carbon structure in this material has a different morphology which appear to be “layers” of carbon deposited on the curved surface of the metal nanoparticles as shown in FIGS. 7A-C. The dark elements in FIG. 7A show discrete silver particles while the high crystallinity of the silver particles is shown in FIG. 7C as evident by the presence of the lattice planes. For the most part, the silver particles are discrete and are interspersed within the carbon structure. FIG. 8 also shows this produced material.
(h) 9 nm silver/carbon composition produced by adding a 44,000-ppm concentration of acetylene, utilizing the process described in Example 1 of the PCT 05/027711 Application. FIG. 9 shows this produced material.
(i) Silver/carbon compositions produced using the processes described in the '858 patent Application. FIGS. 10A-F show these produced materials.
(j) Silver/carbon composition produced by adding a 8,800 ppm concentration of methane, utilizing the process described in Example 1 of the PCT 05/027711 Application. FIGS. 11A-C show this produced material. These Figures show the presence of carbyne and reflect the intertwined layers of carbon with interspersed silver particles. This composition had a specific surface area of 19 m2/g and had a silver and carbon mass content of 98.5/1.5, respectively.
(k) Copper/carbon composition produced by adding a 44,000 ppm concentration of acetylene, utilizing the process described in Example 2 of the PCT 05/027711 Application. FIGS. 12A-D show this produced material. These Figures show the presence graphitic and fullerene carbon. This composition had a specific surface area of 44 m2/g and had a mass content of 20 wt % copper and 80 wt % carbon.
(l) Iron/carbon composition produced by adding a 4,400 ppm concentration of acetylene, utilizing the process described in Example 3 of the PCT 05/027711 Application. FIGS. 13A-B show this produced material. This composition had a specific surface area of 65 m2/g.
(m) Iron/silver/carbon composition/alloy produced by adding acetylene, utilizing the process described in Example 4 of the PCT 05/027711 Application. FIGS. 14A-B show this produced material.

These and other nanomaterials may be obtained from Nanotechnologies, Inc. of Austin, Tex.

The nanomaterials for use as anti-viral agents have discrete metal particles (typically silver), which can be interdispersed within a carbon structure. Often, the carbon structure itself within the nanomaterials contains carbyne structures. TEM images of such silver/carbon composite indicate that there is no coating on the small discrete silver particles. Many processes require surface functionalization, such as a surfactant or dispersant, to keep the particles discreet. Consequently, the silver particles in the carbon matrix will have higher reactive surfaces than other silvers. This appears to be true for the copper, iron, gold and more than likely other metals produced with this process. It is believed that this property of these nanomaterials leads to the materials being anti-viral agents.

### Antiviral Uses

A novel use of the new nanometal materials (such as the silver/carbon compositions) is the use of them as anti-viral agents. For instance for silver, (and more specifically the silver ions that the nanosilver releases) has long been known to have antibacterial properties. Literature indicates that the ions interrupt the bacteria's metabolic functions resulting in termination of the bacteria. Silver has been shown to be ineffective against virus because virus does not have metabolic functions that allow interaction with the silver ions. The new silver/carbon material was tested to determine its virucidal effectiveness. Tests against several viruses were conducted using the American Society for Test Materials (ASTM) test method E1052-96 entitled “Standard Test Method for Efficacy of Antimicrobial Agents Against Viruses in Suspension.” Tests were conducted on viruses which are representative of a broad spectrum of viral families. The tests included large and small variants of RNA and DNA based, and enveloped and non-enveloped viruses. Specifically tests were conducted on Herpes Simplex Virus-1 (HSV-1), Bovine Diarrhea Virus (BVDV; surrogate for human hepatitis C), feline calicivirus (surrogate for Norwalk) and adenovirus.

All tests were conducted with 10e6 CFU (colony forming units) in deionized water at three different concentrations of the 10 nm silver/carbon composition (composition (g) above and as shown in FIGS. 7A-C & 8). Table A below reflects the results of this testing for the different viruses (exposure time of one hour).

TABLE A

| | Silver Composite Material | | | |
|---|---|---|---|---|
| | (10 log reduction of colony | | | |
| | forming units at various conc.) | | | |
| | 10 | 100 | 1000 | Conc. at which host cell |
| Virus | μg/ml | μg/ml | μg/ml | toxicity is observed |
| HSV-1 | 1-2 | 4 | Complete | none |
| BVDV | 0.5-1 | 1-2 | 2-3 | none |
| Feline | 1.5 | 2.2 | 3 | none |
| calicivirus | | | | |
| Adenovirus | 1 | 1.2 | 2 | none |

The data of Table A shows that the silver/carbon composition had significant efficacy against viruses and that the efficacy occurred at levels below toxic levels for the host cells. In other words, at concentrations that inactivate the viruses, the host cells are unaffected. Test results show that the silver/carbon composition interfered in the ability of the virus to replicate using host cells, and essentially terminated the virus.

The results from these tests show that nanosilver composition had an immediate kill of the viruses and at one hour the 1000 μg/ml concentration of the composition had a complete kill of HSV-1. In industry, anti-viral static agents are considered to be materials which prevent growth. These materials typically have at least a Log 0 reduction. Materials are generally considered to have antiviral properties if there is at least a Log 2 reduction and often a Log 3 reduction. Depending on standards, a “complete kill” is defined as between at least a Log 4 or at least a Log 6 reduction. While the current tests were performed for one hour, one skilled in the art will recognize the time sensitivity of these tests. Often, additional kill of the virus will occur with longer exposure times to the anti-viral agent

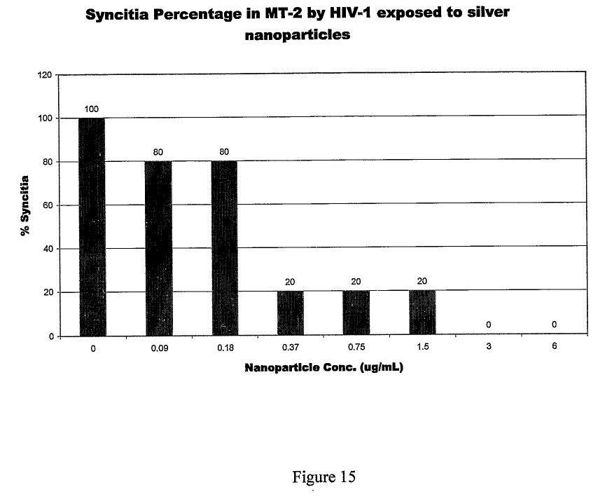

Additional testing was performed on CD4+MT2 cells exposed to the HIV_{iiib} virus (laboratory strain of HIV-1) using the 25 nm silver/carbon composition (composition (e) above and as shown in FIGS. 5A-B). Initial tests were performed to determine the toxicity concentrations against the normal cells and then tests were performed on cells exposed to the virus using material concentrations less than the cytotoxic level. The new material showed complete inactivation of the virus. The syncitia percentage in MT-2 by HIV-1 exposed to the 25 nm silver/carbon composition at varying concentrations is reflected in FIG. 15.

Additional testing was conducted to identify why the nanosilver composition behaves differently than the other silver materials, including, specifically, the silver ions. TEM images indicate that particles in the range of 0-10 nm with predominately icosahedral or dechedral morphology have interacted with the virus. It is believed that these particles tend to be more reactive because of their morphology.

Thus, it appears that smaller nanometal compositions (like nanosilver), such as having an average size of at most about 25 nm, generally exhibit greater anti-viral effectiveness than larger nanometal compositions. And as the average size reduces further, such as at most about 15 nm and then at most about 8 μm, the effectiveness again appears to be generally progressively better. Thus, the ability to form a non-agglomerated nanometal composition (such as by the processes described in the PCT 05/027711 Application and the '858 patent Application) is advantageous.

Tests were also performed to monitor the ionic release of the nanosilver composition. Results showed that there was not a sustained ionic release. Hence, it was believed that the reactive nature of the silver particle's morphology enables the particles to deactivate the virus.

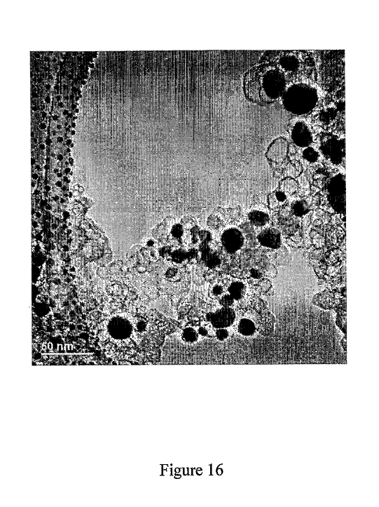

While not intending to be bound by this theory, Applicants further believe that another aspect that may be enabling the nanosilver's anti-viral effectiveness is believed to also be the carbon structure surrounding the silver particles. TEM images and EELS spectrum indicate that in some embodiments the carbon consists of a caged carbyne structures. Analysis suggests that the carbyne structures may be forming cages around the silver particles and keeping them discrete. Experiments show that the particles are ejected from the carbyne structure when they are hit with an electron beam. Additionally, it appears that the carbon structure may act as a type of filter by allowing the smaller particles to be easily ejected or removed while preferentially retaining the larger particles. This may allow the possibility of silver nanoparticles (or other metal nanoparticles) being delivered from the cages to specific sites in an organism. This ejecting effect can be seen in FIG. 16.

In FIG. 16, the particles that were contained within the carbyne structure have moved to the TEM carbon grid. The TEM grid is located on the left side of the image whereas the carbyne structure is the lighter spherical structures in the center of the image.

These results reflect that the nanosilver compositions can be used as an anti-viral agent by subjecting the virus to the composition. Other nanometal materials, such as the copper, copper oxide, iron, cobalt, nickel, and silver oxide are believed to also be effective anti-viral agents.

Incorporating the material into various compounds can produce many different applications. One such product would be to disperse the material at moderate loadings (0.0001-10%) into a solution, such as water or IPA. The solution could then be used as an anti-viral spray to neutralize viruses on surfaces. The solution would be sprayed onto a surface and the liquid would evaporate leaving the nanoparticles on the surface to neutralize any virus. Possible surfaces include but are not limited to countertops, sinks, toilets, wood decking, hospital bed frames, floors, metals, plastics, concrete, rock, masonry, air or liquid filter media, skin and wounds. The material can also be incorporated into a sterile and buffered solution, such as a saline solution, for use as a nose spray, eye drops or inhaler solution to inactivate viruses in the eyes and respiratory system. The material can also be used in products, such as textiles and coatings that may transfer the virus or allow the virus to survive.

By incorporating the material into textiles, plastics, paints, industrial coatings, etc., the nanosilver composition (or other nanometal composition) is available to neutralize the virus. For example, the silver can be dispersed within a latex paint which is then painted onto a surface. The silver within the paint neutralizes the virus. To help facilitate additional activity, the coating may be one that is designed to wear over a given time. This would continually expose particles and would have enhanced performance. For example, the material can be incorporated into a coating such as acrylic latex wax which is applied to a surface and wears off over time. In porous or permeable media such as textiles, the virus may not be on the surface and remain active within the confines of the product. In this application, incorporation throughout the product will neutralize the virus that penetrates into the product. Products would include tissues, bandages, feminine products, diapers, gauze, clothing and fabrics, cleaning sponges, as well as others.

Additionally, the nanometal composition can be incorporated into a cream, lotion, paste or ointment to provide antiviral efficacy. For example, the particles may be incorporated into a petroleum jelly at moderate loading (0.0001-10%). The ointment can then be applied to protect open wounds and sores by forming a protective barrier.

Another application of the nanometal compositions is in the use of internal medicine. In one embodiment of the invention, the silver could be linled to a specific protein or antibody, such as with an aptamer, to enable selective viral efficacy. Another pharmaceutical application is to incorporate the material in a time release drug delivery system, such as Poly(2-hydroxy ethyl methacrylate), Poly(N-vinyl pyrrolidone), Poly(methyl methacrylate), Poly(vinyl alcohol), Poly(acrylic acid), Polyacrylamide, Poly(ethylene-co-vinyl acetate), Poly(ethylene glycol), Poly(methacrylic acid), Polylactides (PLA), Polyglycolides (PGA), Poly(lactide-co-glycolides) (PLGA), Polyanhydrides, Polyorthoesters. This would allow the material to be delivered to a specific location within the body or have a regulated and or controlled release. Additionally systems can be employed that are responsive to changes in the environment such as pH, concentration gradients, temperature, etc. The systems may also be responsive to external stimulus such as ultra sonic action, radiation (X-ray, V, etc.), magnetic fields, temperature changes and electric fields. These systems allow greater control of the materials to enhance the viral efficacy.

All patents and publications referenced herein are hereby incorporated by reference. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

1. A process comprising the steps of:
(a) selecting a composition of generally unagglomerated nanoparticles, wherein the nanoparticles comprise a metal; and
(b) utilizing the nanoparticles as an anti-viral agent.

2. The process of claim 1, wherein the metal comprises silver.

3. The process of claim 1, wherein the metal comprises a metal selected from the group consisting of silver, copper, copper, iron, cobalt, nickel, oxides thereof, and combinations and alloys thereof.

4. The process of claim 1, wherein the composition further comprises a second metal.

5. The process of claim 1, wherein the composition further comprises carbon.

6. The process of claim 5, wherein the carbon comprises carbon in the form of carbyne.

7. The process of claim 1, wherein the composition is in the form of a nanopowder.

8. The process of claim 1, wherein the nanoparticles are formed utilizing a high power, pulsed plasma process

9. The process of claim 8, wherein the high power pulsed process comprises the use a precursor, wherein the precursor comprises carbon.

10. The process of claim 9, wherein the precursor is a gaseous precursor.

11. The process of claim 10, wherein the gaseous precursor is selected from the group consisting of acetylene, methane, and combinations thereof.

12. The process of claim 1, wherein the nanometal has an average size of at most about 25 nm.

13. The process of claim 12, wherein the average size of the nanometal is at most about 15 nm.

14. The process of claim 12, wherein the average size of the nanometal is at most about 8 nm.

15. The process of claim 1, wherein, the composition is utilized as an anti-viral agent at a concentration of at least about 10 μg/ml.

16. The process of claim 15, wherein the concentration is at least about 100 g/ml.

17. The process of claim 16, wherein the concentration is at least about 1000 μg/ml.

18. The process of claim 1, wherein the composition is utilized as an anti-viral agent for at least one hour.

19. The process of claim 1, wherein the composition has at least about a Log 2 reduction after one hour.

20. The process of claim 1, wherein the composition has at least about a Log 3 reduction after one hour.

21. The process of claim 1, wherein the composition has at least about a Log 4 reduction after one hour.

22. The process of claim 1, wherein the composition has at least about a complete kill after one hour.

23. The process of claim 1, wherein the composition is dispersed in a solution.

24. The process of claim 23, wherein composition is loaded in the solution at a concentration between about 0.0001% and 10%.

25. The process of claim 23, wherein the solution is utilized as an anti-viral spray.

26. The process of claim 23, wherein the solution is utilized on a surface selected from the group consisting of countertops, sinks, toilets, wood decking, hospital bed frames, floors, metals, plastics, concrete, rock, masonry, air or liquid filter media, skin and wounds.

27. The process of claim 23, wherein the solution is a sterile and buffered solution.

28. The process of claim 27, wherein the solution is a saline solution.

29. The process of claim 27, wherein the solution is utilized in a use selected from the group consisting of (i) as a nose spray, (ii) as eye drops, (iii) as an inhaler solution to inactivate viruses in eyes, (iv) as an inhaler solution to inactive viruses in a respiratory system, and (v) combinations thereof.

30. The process of claim 1, wherein the composition is utilized in or on a textile, plastic, paint, or industrial coating.

31. The process of claim 1, wherein the composition is utilized as a coating.

32. The process of claim 31, wherein the coating wears out over time.

33. The process of claim 1, wherein the composition is utilized in a material selected from the group consisting of tissues, bandages, feminine products, diapers, gauze, clothing and fabrics, and cleaning sponges.

34. The process of claim 1, wherein the composition is utilized in a product operable for providing anti-viral efficacy and wherein said product is selected from the group consisting of a cream, lotion, paste, ointment, and combinations thereof. 35.

35. The process of claim 34, wherein said product comprises the composition in an amount between about 0.0001% to about 10%.

36. The process of claim 34, wherein the product is applied to open wounds.

37. The process of claim 1, wherein the nanoparticles are used as an anti-viral agent in an internal medicine process.

38. The process of claim 1, wherein the metal is linked to a selected protein or antibody.

39. The process of claim 1, further comprising incorporating the composition in a drug delivery system.

40. The process of claim 39, wherein said drug delivery system includes a material selected from the group consisting of Poly(2-hydroxy ethyl methacrylate), Poly(N-vinyl pyrrolidone), Poly(methyl methacrylate), Poly(vinyl alcohol), Poly(acrylic acid), Polyacrylamide, Poly(ethylene-co-vinyl acetate), Poly(ethylene glycol), Poly(methacrylic acid), Polylactides (PLA), Polyglycolides (PGA), Poly(lactide-co-glycolides) (PLGA), Polyanhydrides, Polyorthoesters, and combinations thereof.

41. The process of claim 39, wherein the drug delivery system comprises at least on of delivering the nanoparticles to a specific treatment location, regulating the release of the nanoparticles, and controlling the release of the nanoparticles.

42. The process of claim 39, wherein the drug delivery system is responsive to a change selected from the group consisting of pH, concentration gradients, temperature, and combinations thereof.

43. The process of claim 39, where the drug delivery system is responsive to external stimulus, wherein said external stimulus is selected from the group consisting of ultra sonication, radiation, magnetic fields, temperature changes and electric fields.

44. The process of claim 1, wherein the nanoparticles is utilized to treat a virus selected from the group consisting of HSV-1, BVDV, Feline calicivirus, Adenovirus, and combinations thereof.

45. The process of any of claim 1 in which selecting a composition includes selecting a composition of generally unagglomerated nanoparticles within carbon structures that keep the nanoparticles from agglomerating.

46. The process of any of claim 1 in which selecting a composition includes selecting a composition that includes metallic nanoparticles interspersed within carbyne structures.

47. The process of any of claim 1 in which selecting a composition includes selecting a composition in which the metallic nanoparticles are kept discreet without coating the particles.

48. The process of any of claim 1 in which selecting a composition includes selecting a composition in which the metallic nanoparticles are kept discreet without having surface functionalization.

49. The process of any of claim 1 in which selecting a composition includes selecting a composition in which the metallic nanoparticles are kept discreet without using a dispersant or surfactant.

50. The process of any of claim 1 in which selecting a composition includes selecting a composition in which carbyne strictures form cages around metallic nanoparticles.

51. The process of any of claim 1 in which selecting a composition includes selecting a composition that includes metallic nanoparticles interspersed within carbyne structures and further comprising ejecting particles from the carbyne structures, the ejected particle providing antiviral activity.
